SEQUENCE LISTING (2 sequences)

SEQ ID NO: 1 - DNA, 21 bp, synthetic construct
  source 1..21: mol_type = other DNA
cagatagagtctatcctgatc

SEQ ID NO: 2 - DNA, 20 bp, synthetic construct
  source 1..20: mol_type = other DNA
gtctgcatgccattgaagtc

(19) United States
(12) Patent Application Publication
(10) Pub. No.: US 2025/0249068 A1
(43) Pub. Date: Aug. 7, 2025

(54) USE OF MICROBIAL HEMEPROTEIN FOR INCREASING MUCIN PRODUCTION IN GUT TRACT

(71) Applicant: HEMOLAB LTD. CO., Seoul (KR)

(72) Inventors: Byung Ah KIM, Seoul (KR); Seung Ki LEE, Seoul (KR); Kyung Hoon PARK, Seoul (KR)

(73) Assignee: HEMOLAB LTD. CO., Seoul (KR)

(21) Appl. No.: 19/042,759

(22) Filed: Jan. 31, 2025

(30) Foreign Application Priority Data
Feb. 2, 2024 (KR) 10-2024-0016692
Apr. 9, 2024 (KR) 10-2024-0047862

(51) Int. Cl. A61K 38/16; A23L 33/18; A61K 35/74; A61P 1/00

(52) U.S. Cl. CPC A61K 38/164; A23L 33/18; A61K 35/74; A61P 1/00

(57) ABSTRACT

The present disclosure relates to a use of microbial hemeproteins for promoting mucin production, in particular, for improving intestinal immunity and treating or preventing enteritis by increasing production of mucin, which forms the intestinal mucus layer.

### CROSS-REFERENCE TO RELATED APPLICATION

This application is based on and claims priority under 35 U.S.C. § 119 to Korean Patent Application No. 10-2024-0047862, filed on Apr. 9, 2024, and Korean Provisional Patent Application No. 10-2024-0016692, filed on Feb. 2, 2024, in the Korean Intellectual Property Office, the disclosure of each of which is incorporated by reference herein in its entirety.

### BACKGROUND

### 1. Field

The present disclosure relates to a use of microbial hemeprotein for promoting mucin production, in particular, a use of microbial hemeproteins for strengthening the intestinal mucosa and for treating or preventing enteritis by increasing production of mucin, which forms the intestinal mucus layer.

### 2. Description of the Related Art

Mucin is a high-molecular-weight, heavily glycosylated protein produced by epithelial tissues, including those of the gastrointestinal tract, lungs, kidneys, ovaries, mammary glands, and pancreas. Under normal physiological conditions, mucin protects epithelial tissues such as the gastric and intestinal mucosa. In particular, mucin present in the intestinal barrier acts as a barrier defending against infection by bacteria or fungi and promotes lubrication and the excretion of waste.

When enteritis, which is primarily caused by bacterial or viral infection, occurs, bacteria belonging to Enterobacteriaceae increase, degrade mucins, and facilitate the invasion of various bacteria into the intestine, thereby inducing inflammation.

The mucus layer and the epithelial cells of the intestine form a physical barrier against toxic and harmful substances present in the intestinal lumen and underlying tissues. In particular, secretory mucus MUC2, which is expressed by goblet cells belonging to the intestinal epithelium, forms an extracellular mucus layer that protects the intestine. In addition, inflammatory substances resulting from colitis correlate with reduced synthesis and secretion of MUC2, and the mucosal barrier is known to play an important role in the progression of the disease (Gastroenterology, 2006, 131:117-129).

Biomass or extracts derived from cells with increased activities in biological processes essential to life phenomena-namely, cells exhibiting highly activated cell division-exhibit high biological activity and thus can be advantageously used as single-cell proteins, which can be collectively referred to as high-vital single-cell proteins (hSCPs). The high-vital single-cell proteins required for such essential biological processes may be a balanced mixture of various complex proteins bound to diverse prosthetic molecules that are involved in in vivo energy-production (e.g., heme iron, quinone, flavin, iron-sulfur complexes, copper atoms, and NAD+), detoxification of reactive oxygen species (e.g., magnesium atoms, heme iron, and flavin), oxidation-reduction balancing (glutathione, flavin, sulfur-containing cysteine, and NAD (P)+), and various environmental sensing and signaling processes (e.g., heme iron, flavin, NAD+, and various vitamins).

Among molecules capable of exhibiting high biological activity in these essential biological processes, one representative example is heme iron, which is distributed in nature in the form of various hemeproteins (also referred to as hemoprotein: heme-conjugated protein) in which the iron acts as a prosthetic group bound to a protein, and which plays an essential role in the energy production processes of living organisms-such as oxygen (O₂) transport, electron transport, and the removal of toxic oxygen species (e.g., catalase and peroxidase)—and in the elimination of reactive oxygen species (ROS). In addition, heme iron, which has a high absorption rate in vivo and causes no side effects, is utilized as a valuable source of iron, and in particular, heme iron produced by microorganisms having heme iron-producing capability is employed in the form of microbial hemoprotein for use as a feed additive or for improving the viability/preservation of lactic acid bacteria.

Korean Patent Application Publication No. 2018-0049612 relates to a composition for promoting the growth or enhancing the preservation of lactic acid bacteria, comprising microbial hemeproteins, and discloses that a microbial hemoprotein extract increases biomass production of lactic acid bacteria and improves shelf life by enhancing viability. However, it does not disclose or suggest use of microbial hemeproteins for promotion of mucin production and prevention or treatment of enteritis.

The inventors of the present disclosure conducted research on the treatment or prevention of obesity by microbial hemeprotein to find that hemeproteins obtained from microbial culture increases mucin production in the intestine, thereby exhibiting excellent effects in preventing or treating enteritis, and thus completed the present disclosure.

### SUMMARY

Provided is a use of a microbial hemeprotein for strengthening the intestinal mucosal layer.

In addition, provided is a use of a microbial hemeprotein for treating or preventing enteritis.

Additional aspects will be set forth in part in the description which follows and, in part, will be apparent from the description, or may be learned by practice of the presented embodiments of the disclosure.

One aspect of the present disclosure provides a composition for strengthening the intestinal mucosa, comprising a microbial hemeprotein.

As used herein, the term “strengthening the intestinal mucosa” refers to strengthening intestinal protection by increasing the production of mucin, a secretory mucus, in the intestinal mucosa, which includes the mucus layer and epithelial cells that function as a physical barrier against toxic and harmful substances.

As used herein, the term “hemeprotein” or “hemoprotein” refers to a protein with bound heme iron and is produced by microorganisms having heme iron-producing capability and accumulated within the microorganism or secreted into the culture medium.

The hemeprotein may be in various forms, such as a microorganism having a high hemeprotein content, a culture of the microorganism, an extract or a high-hemoprotein-content microbial protein (single-cell protein) extracted therefrom. The hemeprotein may include heme iron produced by a microorganism, or a protein or microbial cells containing the heme iron, and because it is derived from a microorganism, the term “microbial hemeprotein” and “microbial hemeprotein extract” are used interchangeably.

As used herein, the term “microorganism having heme iron-producing capability” or “microorganism having a high hemeprotein content” refers to a microorganism that produces heme iron, and accumulates hemeprotein internally or externally during culture, thereby having a higher hemeprotein content compared to microorganisms that lack or have low heme iron-producing capability. In the present specification, the term “microorganism having a high hemeprotein content” is used interchangeably with “microorganism having heme iron-producing capability”. A microorganism having a high hemeprotein content may be one that has been selected by adaptive evolution, without genetic modification, to possess a high growth rate and correspondingly high biosynthetic capacity. For example, the microorganism may be Corynebacterium glutamicum HemoP1 disclosed in Korean Patent No. 2210764 or Klebsiella variicola HemoC1 disclosed in Korean Patent No. 2118083.

In an embodiment of the present disclosure, the microbial hemeprotein may be in the form of a microbial culture obtained by culturing a microorganism having heme iron-producing capability, microbial cells isolated therefrom, or a hemeprotein separated and purified therefrom.

In an embodiment of the present disclosure, the microorganism may be one that has been selected for growth rate and heme iron-producing capability by adaptive evolution to have a higher hemeprotein content than its parent strain, without any genetic modification.

In an embodiment of the present disclosure, the microorganism having heme iron-producing capability or high hemeprotein content may be a microorganism that has been selected for growth rate and heme iron-producing capability by adaptive evolution and thus, without genetic modification, has a higher hemeprotein content compared to the parent strain. Examples may include Corynebacterium glutamicum HemoP1 disclosed in Korean Patent No. 2210764 and Klebsiella variicola HemoC1 disclosed in Korean Patent No. 2118083.

In an embodiment of the present disclosure, the microorganism having a high hemeprotein content may be a microorganism that, without any artificial genetic modification, may be isolated from nature as having naturally increased activity or expression level of enzymes involved in energy metabolism or reactive oxygen species detoxification; for example, a microorganism of the genus Corynebacterium.

In an embodiment of the present disclosure, the microorganism having a high hemeprotein content may be a Corynebacterium glutamicum derived from a healthy gut microbiota that exhibits both high energy metabolism for rapid growth and high resistance to oxidative stress and produces a red pigment upon disruption, thereby indicating that it contains a higher hemeprotein content than common microorganisms.

In an embodiment of the present disclosure, the Corynebacterium glutamicum may be Corynebacterium glutamicum HemoP1 disclosed in Korean Patent No. 2210764.

As used herein, the term “parent strain” refers to the original strain to which modification by recombination, mutagenesis, or adaptive evolution has been made.

In an embodiment of the present disclosure, the microbial hemeprotein may be in the form of a culture of Corynebacterium glutamicum, a dried product of the culture, an extract of the culture, or a hemeprotein extract purified from the culture. Since the culture obtained by culturing Corynebacterium glutamicum contains a hemoprotein produced by Corynebacterium glutamicum, the culture itself may be used as a source of hemoprotein or heme iron; or the culture may be further subjected to a step of extracting hemoerotein or heme iron therefrom to obtain and use a hemeprotein extract or a heme iron extract; or the extracting step is followed by a purification step to obtain and use a hemeprotein or heme iron.

In an embodiment of the present disclosure, the microbial hemeprotein may be in the form of a culture of Klebsiella variicola, a dried product of the culture, an extract of the culture, or a hemeprotein extract purified from the culture. Because the culture obtained by culturing Klebsiella variicola contains a hemeprotein produced by Klebsiella variicola, the culture itself may be used as a source of hemeprotein or heme iron; or the culture may be further subjected to a step of extracting hemeprotein or heme iron therefrom to obtain and use a hemeprotein extract or a heme iron extract; or the extracting step is followed by a purification step to obtain and use a hemeprotein or heme iron.

In an embodiment of the present disclosure, the microbial hemeprotein may be obtained by a method comprising recovering microbial cells from the microbial culture, resuspending and disrupting the recovered microbial cells, and centrifuging or drying the disrupted suspension.

In an embodiment of the present disclosure, the culturing of a microorganism having a high hemeprotein content may be carried out using a medium known in the art to which the present disclosure pertains. The culture method and conditions may be selected by one of ordinary skill in the art to which the present disclosure pertains.

In an embodiment of the present disclosure, the microbial hemeprotein may increase the production of MUC2 (Mucin 2).

In an embodiment of the present disclosure, the content of the microbial hemeprotein in the composition may be 0.01 wt % to 70 wt %, based on the total weight of the composition.

In an embodiment of the present disclosure, the composition may be used as a feed ingredient, a food ingredient, or a health-functional food, and may be used in the form of powder, granule, pill, tablet, capsule, food, beverage, or the like.

In an embodiment of the present disclosure, the composition may be used as a feed additive.

As used herein, the term “feed additive” refers to a substance added to feed for a particular functional or nutritional purpose, and may be administered alone or in combination with feed.

Another aspect of the present disclosure provides a pharmaceutical composition for preventing or treating enteritis, the composition comprising a microbial hemeprotein.

As used herein, the term “enteritis” refers to a disease caused by impairment of the normal barrier function of the intestine and may include abdominal distention, abdominal discomfort, infectious diarrhea caused by pathogenic microorganisms, gastroenteritis, inflammatory bowel disease, neurogenic enteritis syndrome, irritable bowel syndrome, small-intestinal bacterial overgrowth, and rapid intestinal transit diarrhea. Inflammatory bowel disease (IBD) is a disease characterized by abnormal chronic inflammation in the intestinal tract with cycles of remission and relapse. The most common forms are ulcerative colitis and Crohn's disease, and the exact pathogenesis remains unclear.

As used herein, the term “prevention” means inhibiting or delaying the onset of enteritis or the occurrence of its symptoms by administration of a composition according to the present disclosure.

As used herein, the term “treatment” means mitigating, alleviating, or curing enteritis or its symptoms by administration of a composition according to the present disclosure.

The human intestinal tract, which serves as a reservoir for intestinal bacteria, performs the basic functions of digestion, absorption, and excretion. The intestinal mucosa has various immunological functions, including a mucosal barrier that blocks the entry of external substances such as intestinal microorganisms, their byproducts, antigens, and toxins into the bloodstream, as well as immunoglobulin secretion and formation of a defense system including macrophages. Intestinal mucosal cells maintain intact intercellular spaces; however, when tight junctions between cells are weakened by stimulation or damage, intestinal mucosal permeability to macromolecules in circulation increases, which may lead to entry of external substances such as pathogens and antigens, triggering an inflammatory response. In the colon, the mucus or mucosal layer serves as a protective barrier against exposure to intestinal microorganisms. Exposure of epithelial cells to intestinal microorganisms can cause enteritis, and mucin (MUC2) is known to play a major role in the colonic mucosal layer (Proc Natl Acad Sci USA 2008; 105:15064-15069) Studies on the relationship between the mucus barrier and ulcerative colitis have shown a significant decrease in MUC2 levels in ulcerative colitis, with evidence indicating that increased MUC2 expression is effective in alleviating or treating ulcerative colitis (Korean J Gastroenterol Vol. 73, No. 5, 311-312).

It has been found that microbial hemeprotein increases mucin production in the intestine, which exerts a preventive or therapeutic effect on inflammatory bowel conditions such as colitis, ulcerative colitis, and irritable bowel syndrome.

This increase in mucin production has beneficial effects, including protection of the intestinal mucosa, promotion of defecation, and enhancement of lactic acid bacteria colonization and proliferation, thereby preventing, alleviating, or treating enteritis.

In an embodiment of the present disclosure, the microbial hemeprotein extract may be in the form of a microbial culture obtained by culturing a microorganism having heme iron-producing capability, microbial cells isolated therefrom, or hemeprotein separated and purified therefrom.

In an embodiment of the present disclosure, the enteritis may be ulcerative colitis or irritable bowel syndrome.

In an embodiment of the present disclosure, the pharmaceutical composition may further comprise an active agent having therapeutic efficacy against enteritis.

In an embodiment of the present disclosure, the composition may be administered in an amount corresponding to 0.0001 mg/kg per day to 100 mg/kg per day, and preferably, 0.001 mg/kg per day to 100 mg/kg per day, of the microbial hemeprotein. The preferred dosage of the microbial hemeprotein of the present disclosure may be appropriately selected by one of ordinary skill in the art based on the subject's condition, body weight, degree of obesity, formulation of the composition, and the route and duration of administration. The composition may be administered once a day or in divided doses per day.

In an embodiment of the present disclosure, the pharmaceutical composition may comprise a pharmaceutically acceptable carrier. The carrier may be an excipient, a diluent, or an adjuvant, but is not limited thereto. The carrier may be selected from the group consisting of lactose, dextrose, sucrose, sorbitol, mannitol, xylitol, erythritol, maltitol, starch, acacia gum, alginate, gelatin, calcium phosphate, calcium silicate, cellulose, methylcellulose, polyvinylpyrrolidone, water, physiological saline, buffer solutions such as phosphate-buffered saline (PBS), methylhydroxybenzoate, propylhydroxybenzoate, talc, magnesium stearate, glycine, histidine, serine, polysorbate, and mineral oil.

In an embodiment of the present disclosure, the pharmaceutical composition may be formulated into any dosage form using conventional methods. The pharmaceutical composition may be formulated, for example, as an oral dosage form (e.g., powder, tablets, capsules, syrup, pills, or granules) or a parenteral dosage form (e.g., injections). Furthermore, the pharmaceutical composition may be prepared as a formulation for systemic or topical delivery.

In an embodiment of the present disclosure, the pharmaceutical composition may be administered orally or parenterally (e.g., intravenously, subcutaneously, intraperitoneally, or topically). The dosage varies according to the subject's condition and weight, disease severity, dosage form, and the route and timing of administration, and may be appropriately selected by one of ordinary skill in the art.

A composition comprising a microbial hemeprotein according to an embodiment of the present disclosure increases mucin production in the intestine, enhances intestinal immune function by increasing the proportion of goblet cells, and is effective in preventing and treating enteritis.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 shows the effect of supplementation with a microbial hemeprotein (hSCP) according to an embodiment of the present disclosure, on the tissue structure of skin tissue obtained from mice fed with a normal diet (ND) or a high-fat diet (HFD). The skin tissue was fixed with 4% paraformaldehyde, stained with H&E, and then observed under a microscope at 100× magnification.

FIG. 2 shows the effect of supplementation with a microbial hemeprotein (hSCP) according to an embodiment of the present disclosure, on the tissue structure of colon tissue obtained from mice fed with a high-fat diet. The colon tissue was stained with AB-PAS, with goblet cells stained blue. A shows tissue from the proximal portion of the colon, and B shows tissue from the distal portion of the colon. The notation “‘(prime)” indicates tissue from mice that were fed supplemented with the microbial hemeprotein, and microscopic observation was performed at 400× magnification.

FIGS. 3 and 4 respectively show the effect of supplementation with a microbial hemeprotein according to an embodiment of the present disclosure, on mucin (MUC2) gene expression in the colon tissue of mice fed with a normal diet (N) or a high-fat diet (H).

In the drawings, N and H respectively indicate a normal diet (normal-calorie diet) and a high-fat diet, 2 W and 4 W respectively indicate 2-week and 4-week feeding periods, CloseColon and DistalColon respectively indicate the proximal colon and the distal colon, _C and _H respectively indicate an hSCP 0% control group and an hSCP 0.05% supplementation group, and ns and */*** respectively indicate (not statistically significant) and p<0.05/0.001.

### DETAILED DESCRIPTION

Reference will now be made in detail to embodiments, examples of which are illustrated in the accompanying drawings, wherein like reference numerals refer to like elements throughout. In this regard, the embodiments may have different forms and should not be construed as being limited to the descriptions set forth herein. Accordingly, the embodiments are merely described below, by referring to the figures, to explain aspects of the description.

### Example 1. Preparation of Microbial Hemeprotein Extract

A single colony of a Corynebacterium glutamicum HemoP1 (Korean Patent No. 10-2210764) strain or a Klebsiella variicola HemoC1 (Korean Patent No. 10-2118083) strain, each confirmed to have a high microbial hemeprotein content, was inoculated into a test tube containing 15 mL of YS medium (0.5% w/v yeast extract, 1% w/v soytone, and 1% w/v glucose), subjected to shaking culture at 30° C. and 250 rpm for 16 hours, and then transferred to a 5 L jar fermentor containing 3 L of the same medium. After culturing at 30° C. for 48 hours with aeration at 0.5 vvm and agitation at 250 rpm, the obtained culture was centrifuged at 3,000 g for 15 minutes at 4° C. to recover cells, and the recovered cells were washed twice with distilled water. Then, the recovered cells were suspended in 100 mL of distilled water and then disrupted by passing three times through a high-pressure homogenizer (EmulsiFlex-C3, Sonic Corp., Stratford, CT, USA) operated at 15,000 psi to release the hemeproteins contained within the cells. After disruption, the suspension was dried in an oven at 105° C. for 24 hours to obtain the microbial hemeprotein.

### Example 2. Strengthening of the Intestinal Mucosal Layer by Hemeprotein Supplementation in Mice with Diet-Induced Obesity

A high-fat diet, known to cause obesity, promotes intestinal inflammation, and this inflammation is known to be associated with weight gain and insulin resistance (see PLOS One 5, e12191 (2010), and Current Opinion in Clinical Nutrition and Metabolic Care 14, 328-333 (2011)). In the present example, the effect of hemeprotein intake on the intestinal mucosa was investigated in mice with obesity induced by a high-fat diet.

### 2-1. Rearing of Mice

All mouse experiments were conducted at the Dongnam Chemical Research Institute (Animal Facility Registration No. 412, Busan, Republic of Korea). The test animals were male C57BL/6 mice (5 weeks old) obtained from HanaBio (Republic of Korea), which were acclimated and reared for one week prior to experimentation. During rearing, the lighting was maintained on a 12-hour cycle (07:00-19:00), and feed and water were provided ad libitum.

All experiments were conducted in accordance with the guidelines and regulations of the Animal Ethics Committee of Dongnam Chemical Research Institute.

Body weight, feed intake, and water consumption were measured at 3-day intervals throughout the experiment.

The normal diet group (ND) was fed the standard feed AlN 93G ad libitum for 2 or 4 weeks, depending on the group, while the normal diet+hemeprotein group was fed AlN 93G with 0.05% hemeprotein (hSCP) ad libitum for 2 or 4 weeks.

The high-fat diet group (HFD) was fed a 60% HFD (high-fat diet) for 8 weeks, and afterward, the high-fat diet+hemeprotein 10-week group was fed a 60% HFD with 0.05% hemoprotein (HFD10+H2) ad libitum for an additional 2 weeks, and the high-fat diet+hemoprotein 12-week group was fed a 60% HFD with 0.05% hemeprotein (HFD12+H4) ad libitum for an additional 4 weeks.

Table 1 below shows the composition of the 60% HFD provided to the mice.

TABLE 1

HFD (D12492)

| Component | g | kcal |
|---|---|---|
| Casein, 30 mesh | 200 | 800 |
| L-cysteine | 3 | 12 |
| Corn starch | 0 | 0 |
| Maltodextrin 10 | 125 | 500 |
| Sucrose | 68.8 | 275 |
| Cellulose, BW 200 | 50 | 0 |
| Soybean oil | 25 | 225 |
| Lard | 245 | 2205 |
| Mineral mix S10026 | 10 | 0 |
| Dicalcium phosphate | 13 | 0 |
| Calcium carbonate | 5.5 | 0 |
| Potassium citrate | 16.5 | 0 |
| monohydrate | | |
| Vitamin mix V10001 | 10 | 40 |
| Choline bitartrate | 2 | 0 |
| FD&C Red Dye #40 | 0 | 0 |
| FD&C Blue Dye #1 | 0.05 | 0 |
| FD&C Yellow Dye #40 | 0 | 0 |
| Total | 773.85 | 4057 |

Table 2 summarizes the grouping of mice according to the diet.

TABLE 2

| | | | Hemeprotein (H)- |
|---|---|---|---|
| Group | Mouse | Diet | Treatment Period |
| ND2 | 3 | ^{a}ND | Week 0 |
| ND2 + H2 | 3 | ND + 0.05% hSCP | Week 2 |
| ND4 | 3 | ND | Week 0 |
| ND4 + H4 | 3 | ND + 0.05% hSCP | Week 4 |
| HFD10 | 3 | ^{b}HFD12492 | Week 0 |
| HFD10 + H2 | 3 | HFD12492 + 0.05% hSCP | Week 2 |
| HFD12 | 3 | HFD12492 | Week 0 |
| HFD12 + H4 | 3 | HFD12492 + 0.05% hSCP | Week 4 |

^{a}ND: AlN-93G(D10012G), normal (standard) growing rodent diet (ref. https://doi.org/10.1016/j.nutres.2016.06.015)
^{b}HFD: HFD12492, 60% kcal high-fat diet (Research Diet, Inc, New Brunswick, NJ, USA).

The number indicated next to the group name represents the feeding period (weeks).

### 2-2. Body Weight and Body Fat

### (1) Body Weight

The body weights of the normal diet group (ND) and the high-fat diet group (HFD), with or without supplementation of hemeprotein, were measured at 3-day intervals and compared. Starting from Week 2, a decrease in body weight was observed in the HFD group that was fed with hemeprotein supplementation, and when hemeprotein was supplemented, weight gain decreased in both the normal diet group and the high-fat diet group.

Table 3 below shows the results of body weight measurements for each group.

TABLE 3

| | | Body weight (g) | | |
|---|---|---|---|---|
| Group | Mouse | Initial | Final | Increase |
| ^{a}ND2 | 3 | 18.123 ± 0.395 | 23.535 ± 1.640 | 2.710 ± 1.603 |
| ND2 + H2 | 3 | 18.115 ± 0.174 | 21.510 ± 1.896 | 1.029 ± 1.564 |
| ND4 | 3 | 18.062 ± 0.604 | 28.166 ± 1.414 | 7.227 ± 0.904 |
| ND4 + H4 | 3 | 18.104 ± 0.547 | 26.727 ± 1.838 | 5.892 ± 1.297 |
| ^{b}HFD10 | 3 | 18.080 ± 0.344 | 45.861 ± 3.944 | 25.754 ± 3.168 |
| HFD10 + H2 | 3 | 18.062 ± 0.525 | 43.304 ± 8.625 | 22.718 ± 7.698 |
| HFD12 | 3 | 18.083 ± 0.615 | 47.042 ± 1.614 | 26.489 ± 2.227 |
| HFD12 + H4 | 3 | 18.097 ± 0.118 | 44.618 ± 2.164 | 24.326 ± 2.601 |

Values are presented as the mean ± standard deviation in body weight (g) for each group (n = 3). The number indicated next to the group name represents the feeding period (weeks).
^{a}ND: AlN-93G(D10012G), normal (standard) growing rodent diet (ref. https://doi.org/10.1016/j.nutres.2016.06.015)
^{b}HFD: HFD12492, 60% kcal high fat-diet (Research Diet, Inc, New Brunswick, NJ, USA).

### (2) Body Fat

### Collection of Biological Samples

After completion of the mouse rearing, body fat was measured in the normal diet (ND) and high-fat diet (HFD) groups, with and without hemeprotein supplementation.

Mice were anesthetized with CO₂ gas and sacrificed, and blood samples were collected from the abdominal aorta. The blood samples were maintained at 18° C. for 30 minutes and then centrifuged (1,200×g, 15 minutes, 4° C.) to separate the serum. The serum samples were stored in an ultra-low-temperature freezer until analysis.

Then, the intestine (large intestine and cecum) and skin were excised and examined, and were fixed with a 4% paraformaldehyde solution for histological analysis.

### Serum Triglycerides (TG) and Total Cholesterol (T-chol)

Serum triglycerides (TG) and total cholesterol (T-cholesterol) were measured using an automatic analyzer (model 760011; Hitachi, Japan) at the Korea Non-clinic Test Support Center (KNCTSC, Korea).

It was found that the microbial hemeprotein supplementation resulted in generally lower levels of triglycerides and total cholesterol in both the normal diet (ND) and high-fat diet (HFD) groups than those in the groups without the supplementation.

These results are shown in Table 4 below.

TABLE 4

| | | Triglycerides (TG: | Total Cholesterol (TC: |
|---|---|---|---|
| Group | Mouse | mg/mL) | mg/mL) |
| ^{a}ND2 | 3 | 166 ± 35 | 138 ± 18 |
| ND2 + H2 | 3 | 135 ± 88 | 127 ± 1 |
| ND4 | 3 | 71 ± 13 | 73 ± 23 |
| ND4 + H4 | 3 | 69 ± 11 | 60 ± 15 |
| ^{b}HFD10 | 3 | 217 ± 99 | 231 ± 11 |
| HFD10 + H2 | 3 | 142 ± 15 | 236 ± 43 |
| HFD12 | 3 | 191 ± 16 | 223 ± 32 |
| HFD12 + H4 | 3 | 158 ± 49 | 216 ± 6 |

Values are presented as the mean ± standard deviation for each group (n = 3). The number indicated next to the group name represents the feeding period (weeks).
^{a}ND: AlN-93G(D10012G), normal (standard) growing rodent diet (ref. https://doi.org/10.1016/j.nutres.2016.06.015)
^{b}HFD: HFD12492, 60% kcal high-fat diet (Research Diet, Inc, New Brunswick, NJ, USA).

### Histology—Subcutaneous Fat

The skin tissue, once excised from the mouse, was fixed in 4% paraformaldehyde solution, embedded in a paraffin block, and sectioned. Following deparaffinization, the sections were dehydrated, stained using the hematoxylin & eosin (H&E) method (ab245880, Abcam Inc., Cambridge, UK), and examined under a microscope (E600, Nikon Inc., Japan) at 100× magnification.

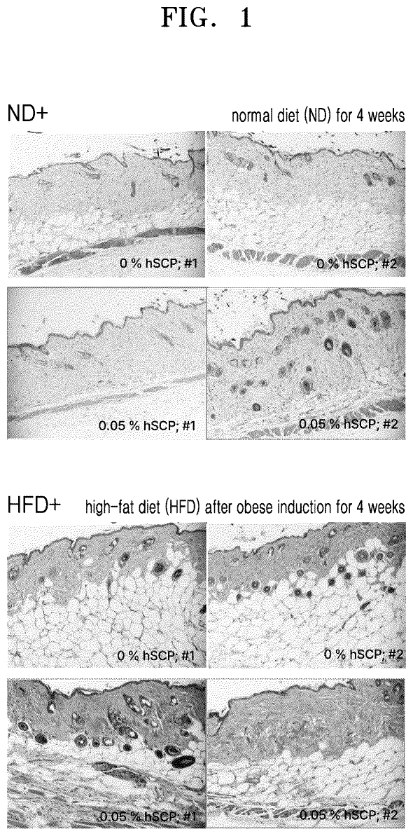

FIG. 1 shows the results observed by optical microscopy after H&E staining of the skin tissue.

Subcutaneous fat in the skin tissue was reduced in both the normal diet group (ND) and the high-fat diet group (HFD) when microbial hemeprotein was supplemented, and a decrease in subcutaneous fat thickness was observed.

### (3) Effect of Microbial Hemeprotein Supplementation

Supplementation with microbial hemeprotein (hSCP or H) was shown to reduce weight gain and fat accumulation in mice from both normal diet group mice and high-fat diet group.

In all analyses, statistical tests were performed using the Starview (ver. 5.0.1) statistical program, and the measured values were expressed as mean±standard deviation. Statistical significance for each analysis was determined by Student's t-test and ANOVA, followed by Tukey's post hoc test.

### 2-3. Mucin Production in Colon Tissue

A high-fat diet is known to promote intestinal inflammation, thereby inducing obesity and insulin resistance (see Current Opinion in Clinical Nutrition and Metabolic Care 14, 328-333 (2011)). In addition, the intestinal mucus layer and epithelial cells form a physical barrier against toxic and harmful substances. In particular, the secretory mucin protein MUC2, which is expressed by goblet cells of the intestinal epithelium, forms an extracellular mucus layer that protects the intestine, and it has been reported that inflammatory substances associated with colitis correlate with decreased synthesis and secretion of MUC2 (see Gastroenterology 2006; 131:117-129).

Accordingly, MUC2 levels in colon tissue were measured in mice in normal diet groups or high-fat-diet groups, with or without supplementation of microbial hemeprotein.

### (1) Histological Analysis

To isolate the intestine, the large intestine and cecum excised from mice in Example 2-2 (1) were cooled on ice and gently washed with 10 mL of distilled water using a 5-ml syringe. The washed intestinal tissue was then placed in a stabilization tube (Microbial Collection and Stabilization Kit, OM-200, Ontario, Canada) for additional examination. The colon tissue was sectioned at 5-mm intervals and immersed in Methacarn solution (60% anhydrous methanol, 30% chloroform, and 10% glacial acetic acid) for 24 hours, and then transferred to a neutral buffer solution (10% buffer, pH 7.0). The colon tissue samples were further dehydrated, cleared, and embedded in paraffin, and were then sectioned to a thickness of 5 μm at the facilities of KNTSC (Korea Non-clinical Technology Solution Center, Seongnam, Gyeonggi, Korea). For mucin visualization, the deparaffinized samples were stained with AB-PAS according to the manufacturer's protocol (ab245876, Abcam Inc.) and examined under an optical microscope.

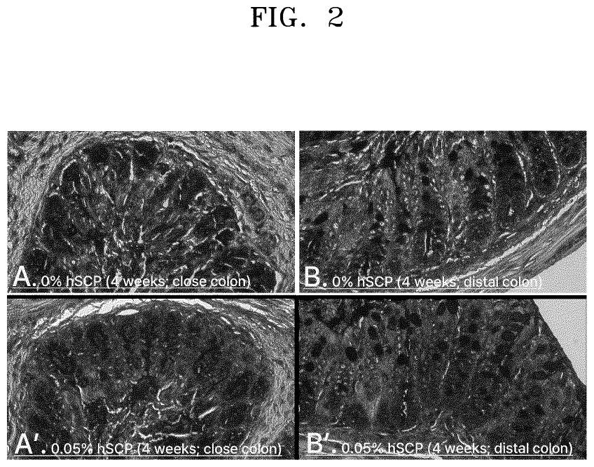

The results are shown in FIG. 2.

The resected colon tissue showed an increased distribution of goblet cells that produce the mucus layer in the intestine. This finding indicates that the hemeprotein diet led to increased mucin content in the intestinal tract. Compared to the control group without hemeprotein supplementation (A and B), mice supplemented with hemeprotein (0.05% hSCP) (A′ and B′) showed stronger staining of goblet cells (blue: mucin-producing cells) in both the proximal and distal colon. This histological observation suggests that mucin production increased in the intestinal tract of mice supplemented with microbial hemeprotein.

It was found that in the high-fat diet group supplemented with hemeprotein, more goblet cells were expressed, leading to elevated mucin production.

### (2) MUC2 Expression Level

As found in (1), the mice supplemented with microbial hemeprotein exhibited elevated mucin production. Real-time PCR was performed to measure the expression level of the MUC2 gene, which is involved in mucin biosynthesis in colon tissue.

Specifically, mRNA of the mucin gene (MUC2) was quantified by PCR. Total RNA was isolated from the proximal colon (located following the small intestine) and distal colon (located just prior to the anus) using TRIzol, according to the manufacturer's instructions. cDNA synthesis was performed using 1 μg of RNA with the PrimerScript 1st strand kit (Takara, Japan). Relative expression was calculated by the 2-(ΔΔCt) method (Livak and Schmittgen, 2001; Methods, 25:402-408 (2001)).

The qRT-PCR primer sequences were as follows:

Mouse_MUC2_F2
(CAGATAGAGTCTATCCTGATC: SEQ ID NO: 1)
Mouse_MUC2_R2
(GTCTGCATGCCATTGAAGTC: SEQ ID NO: 2).

The mRNA concentration of GAPDH was used as a control, and qRT-PCR was performed using Premix Ex Taq (Takara, Japan) with an annealing temperature of 60° C.

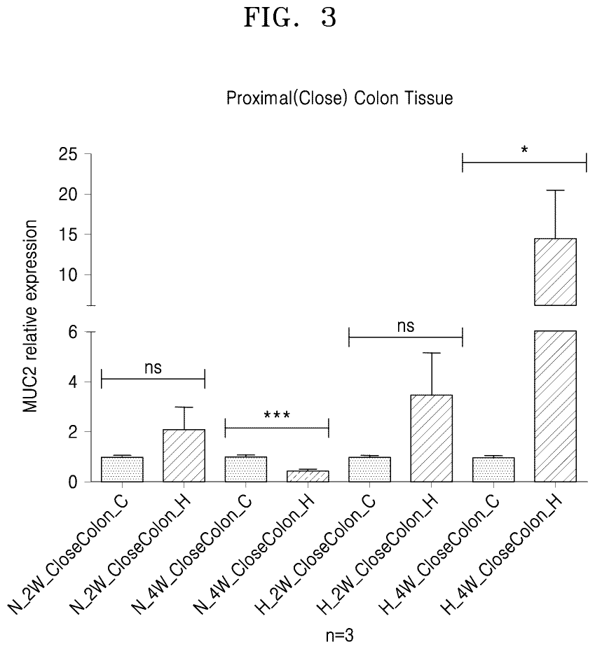

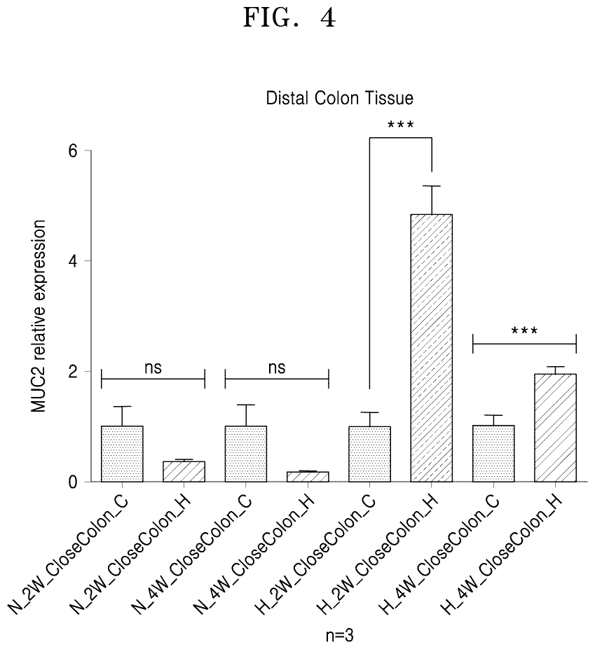

FIGS. 3 and 4 show, respectively, the relative MUC2 expression levels measured in the proximal and distal colon.

In colon tissue obtained from mice fed a normal diet, the difference in MUC2 expression between the control group not supplemented with microbial hemeprotein (hSCP 0.05%) and the group supplemented with the microbial hemeprotein was not statistically significant or was similar in level. However, a marked increase in MUC2 expression was observed in tissue from the high-fat diet group mice, and after 4 weeks of feeding, the MUC2 level in the proximal colon was 15-fold higher than that in the normal diet group (p<0.05). In the distal colon, the MUC2 level was 5-fold higher after 2 weeks (p<0.001) and 2-fold higher after 4 weeks (p<0.001).

While a high-fat diet reduced MUC2 levels in the mouse colon, supplementation with microbial hemeprotein increased MUC2 expression. These results demonstrate that microbial hemeprotein strengthens the colonic mucus barrier and exhibits preventive or therapeutic effects against enteritis.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

1. A method for strengthening the intestinal mucosa in a subject in need thereof, comprising administering a microbial hemeprotein to the subject.

2. The method of claim 1, wherein the microbial hemeprotein is in the form of a microbial culture obtained by culturing a microorganism having heme iron producing capability, microbial cells isolated therefrom, or a hemeprotein separated and purified therefrom.

3. The method of claim 2, wherein the microorganism is a microorganism that has been selected for growth rate and heme iron producing capability by adaptive evolution to have a higher hemeprotein content than its parent strain, without any genetic modification.

4. The method of claim 2, wherein the microbial hemeprotein is obtained by a method comprising recovering microbial cells from the microbial culture, resuspending and disrupting the recovered microbial cells, and centrifuging or drying the disrupted suspension.

5. The method of claim 1, wherein the microbial hemeprotein increases production of Mucin 2 (MUC2).

6. The method of claim 1, wherein the composition is administered as a feed ingredient, a food ingredient, or a health supplement.

7. The method of claim 1, wherein the subject is a patient in need of treating enteritis and the method is used for the treatment of enteritis.

8. (canceled)

9. The method of claim 7, wherein the enteritis is necrotizing enteritis or irritable bowel syndrome.

10. (canceled)
